(19) United States
(12) Patent Application Publication
(10) Pub. No.: US 2011/0123639 A1
(43) Pub. Date: May 26, 2011

(54) Compound for improving brain functioning and/or treatment of brain disorders

(76) Inventors: Agnes Sui-yin CHAN, Hong Kong (CN); Dejian Shi, Dengfeng (CN)

(21) Appl. No.: 12/626,624

(22) Filed: Nov. 26, 2009

(51) Int. Cl. A61K 36/756; A61K 35/12; A61P 25/00; A61P 25/28; A61P 25/08

(52) U.S. Cl. 424/522; 424/769

(57) ABSTRACT

This invention involves a compound that is used for improving brain functioning and/or treatment of brain disorders, made from the following raw materials: (1) one or more medicinal herbs from borax, radix isatidis, honeysuckle, fritillaria cirrhosa D. Don and thunbergii; (2) One or more medicinal herbs from dryobalanops aromatica gaertn. f., calculus bovis, moschus moschiferus L., liquidambar orientalis mill. and gum benzoin; (3) One or more medicinal herbs from cortex phellodendri, coptis chinensis Franch, scutellaria baricalensis, radix gentianae, sophora flavescens; (4) One or more medicinal herbs from baphicacanthus Rhizome and Root, calculus bovis, indigowoad leaf, taraxacum officnala, rhizome of the manyleaf Paris, wild chrysanthemum; and the free selection of (5) Chrysanthemum, folium mori, mint, arctium lappa. This invention also includes the manufacturing method, uses, and the medicinal preparation of the above compounds.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

The brain is housed inside the human skull. It is an extremely important organ that controls an individual's thinking, intelligence, memory, language, motor functions, sensations, emotions, and behavior. When the brain is attacked by diseases, brain functioning will be affected, and the individual will exhibit abnormalities in intelligence, emotions, and behavior.

Brain disorders affect people of all ages, including adults, the elderly, and children. Common brain disorders include epilepsy and tumor for adults; Alzheimer's disease. Parkinson's disease and stroke for the elderly; and ASD and ADHD for children. No matter what etiology the brain disorders stem from, a brain with disorder will lead to impaired functioning, which in turn will have an impact on intelligence and/or emotions. Although there are over a billion people having brain disorders worldwide, there is not yet medication that can completely cure brain disorders.

Norepinephrine in the brain is a mono-amine neural transmitter. Its effect on the brain mainly includes attention and memory, and to a certain degree, emotions. Thus, if the level of norephinephrine in the brain is low, problems with attention, memory and motions will appear. Some common brain disorders such as ADHD is related to the level of norepinephrine. In the West, medications such as Ritalin treats ADHD through increasing norepinephrine level, but there are adverse side effects to these medications. Also, abnormal norepinephrine levels are also related to Alzheimer's disease, in which patients exhibit impairments in attention, memory, and emotional control. Tricyclic Anti-depressants (TCAs) is a class of medication that are used for increasing norepinephrine level in the brain, but they have adverse side-effects.

At present, it is known that some herbal medicine has the effect of improving brain functioning, such as Rhodiola, reported in U.S. Pat. No. 6,399,116, that can increase perfusion and improve work efficiency, memory and attention. Some ancient Chinese medical texts and contemporary research reports also showed that some “orifice-opening” Chinese compound formulae that have been used to treat brain disorders. For example,

(1) Angong Niuhuang Boluses (Wen Bing Tiao Ban), pills, contains 30 each of calculus bovis, rhinoceros horn, chinabar, coptis chinensis franch, scutellaria baricalensis, gardenia jasminoithes ellis, realgar, curcuma aromatica salisb; 15 g of hyriopsis cumingii Lea; 8 g each of moschus moschiferus L. and dyobalanops aromatica gaertn. f.
(2) Zixuedan (Wai Tai Mi Yao), consisting of 1500 g each of gypsum, calcitum, gypsum rubrum, talc, magnetite; 150 g each of rhinoceros horn, saiga tatarica L.; 150 g each of aristolochia shimadai and Chinese eaglewood; 500 g each of radix scrophulariae, rhizoma cimicifugae; 240 g of radix glycyrrhiza; 30 g of syringa oblate; 5000 g of mirabilite; 96 g of niter; 1.5 g of moschus moschiferus L.; 90 g of chinabar.
(3) Guanxin Suhe Wa (Pharmacopoeia of the People's Republic of China, 2000 edition), consisting of 50 g of liquidambar orientalis mill.; 105 g of dryobalanops aromatica gaertn. f.; 105 g of boswellia carteri; 210 g of santalum album, 210 g of aristolochia shimadai.

This invention uses different herbal compounds from the above well-known herbal formulae to increase the norepinephrine level in the brain, in order to improve brain activity and symptoms resulting from brain disorders.

### BRIEF SUMMARY OF THE INVENTION

This invention involves a compound that is used for improving brain functioning and/or treatment of brain disorders, made from the following raw materials: (1) one or more medicinal herbs from borax, radix isatidis, honeysuckle, fritillaria cirrhosa D. Don and thunbergii; (2) One or mere medicinal herbs from dryobalanops aromatica gaertn. f., calculus bovis, moschus moschiferus L., liquidambar orientalis mill. and gum benzoin; (3) One or more medicinal herbs from cortex phellodendri, coptis chinensis Franch, scutellaria baricalensis, radix gentianae, sophora flavescens; (4) One or more medicinal herbs from baphicacanthus Rhizome and Root, calculus bovis, indigowoad leaf, taraxacum officnala, rhizome of the manyleaf Paris, wild chrysanthemum; and the free selection of (5) Chrysanthemum, folium mori. mint, arctium lappa. This invention also includes the manufacturing method, uses, and the medicinal preparation of the above compounds.

### BRIEF DESCRIPTION OF THE SEVERAL VIEWS OF THE DRAWING

Not applicable.

### DETAILED DESCRIPTION OF THE INVENTION

In the first instance, this invention provides a health promotion and medicinal compound produced from herbs, that through increasing the norepinephrine level in the brain, acts to improve different symptoms as a result of brain disorders.

Specifically, this invention that is used for improving brain functioning and/or treatment of brain disorders is produced from the following raw materials:
(1) One or more medicinal herbs from borax, radix isatidis, honeysuckle, fritillaria cirrhosa D. Don and thunbergii;
(2) One or more medicinal herbs from dryobalanops aromatica gaertn. f., calculus bovis, moschus moschiferus L., liquidambar oriental is mill. and gum benzoin;
(3) One or more medicinal herbs from cortex phellodendri, coptis chinensis franch, scutellaria baricalensis, radix gentianae, sophora flavescens;
(4) One or more medicinal herbs from baphicacanthus rhizome and root, calculus bovis, indigowoad leaf, taraxacum officnala, rhizome of the manyleaf Paris, wild chrysanthemum; and the free selection of
(5) Chrysanthemum, folium mori, mint, arctium lappa.

In the second instance, this invention includes the compound described in the first instance above and medically acceptable formatives.

In the third instance, this invention provides the manufacturing method, which includes the grinding to powder form and mixing together of the raw materials, extraction by water, and filtered. The weight ratio of the raw materials and water is 1:20-200.

In the fourth instance, this invention provides the uses of the compound as described in the first instance in improving brain functioning and/or treatment of brain disorders.

### Procedure

### Compounds

As described above, this invention provides a health promotion and medicinal compound produced from herbs, that increases the norepinephrine level in the brain which in turn increases brain activity, resulting in improving different symptoms as a result of brain disorders.

Specifically, this invention that is used for improving brain functioning and/or treatment of brain disorders is produced from the following raw materials:
(1) One or more medicinal herbs from borax. radix isatidis, honeysuckle, fritillaria cirrhosa D. Don and thunbergii;
(2) One or more medicinal herbs from dryobalanops aromatica gaertn. f., calculus bovis, Moschus moschiferus L., Liquidambar orientalis Mill. and gum benzoin;
(3) One or more medicinal herbs from cortex phellodendri, coptis chinensis franch, scutellaria baricalensis, radix gentianae, sophora flavescens;
(4) One or more medicinal herbs from baphicacanthus rhizome and root, calculus bovis, indigowoad leaf, taraxacum officnala, rhizome of the manyleaf Paris, wild chrysanthemum; and the free selection of
(5) Chrysanthemum, folium mori, mint, arctium lappa.

Preferably, taking the total weight of raw materials (1) to (5) as 100%, the respective percentage weight of the raw materials (1), (2), (3), (4) and (5) are 10-80%, 3-40%, 3-40%, 3-40%, and 0-50%; more preferably, the percentage weight should respectively be 30-80%, 5-20%, 3-20%, 3-20%, and 0-40%; the most preferred percentage weight are 40-75%, 6-15%, 3-10%, 3-10%, and 0-30%.

In one preferred method, the described compound is produced from borax, dryobalanops aromatica gaertn. f., cortex phellodendri, coptis chinensis franch and baphicacanthus rhizome and root. Taking the total weight of the raw materials as 100%, the usage of the different raw materials are as follows: borax 20-80%, dryobalanops aromatica gaertn. f. 5-30%, cortex phellodendri 3-20%, coptis chinensis franch 3-20%, and baphicacanthus rhizome and root 3-20%; the usage in another preferred method are: borax 50-80%, dryobalanops aromatica gaertn. f. 7-20%, cortex phellodendri 5-20%, coptis chinensis franch 3-15%, and baphicacanthus rhizome and root 3-15%; the usage in the most preferred method are: borax 60-77%, dryobalanops aromatica gaertn. f. 10-15%, cortex phellodendri 5-10%, coptis chinensis franch 4-8%, and baphicacanthus rhizome and root 4-8%.

In another preferred method, the described compound is produced from borax, dryobalanops aromatica gaertn. f., cortex phellodendri, coptis chinensis franch, baphicacanthus rhizome and root and chrysanthemum. Taking the total weight of the raw materials as 100%, the usage of the different raw materials are as follows: borax 10-70%, dryobalanops aromatica gaertn. f. 5-30%, cortex phellodendri 5-30%, coptis chinensis franch 5-30%, baphicacanthus rhizome and root 5-30%, and chrysanthemum 5-70%; the usage in another preferred method are: borax 20-60%, dryobalanops aromatica gaertn. f. 5-20%, cortex phellodendri 5-20%, coptis chinensis franch 5-20%, baphicacanthus rhizome and root 5-20%, and chrysanthemum 10-50%; the usage in the most preferred method are: borax 30-50%, dryobalanops aromatica gaertn. f. 5-15%, cortex phellodendri 5-15%, coptis chinensis franch 5-15%, baphicacanthus rhizome and root 5-15%, and chrysanthemum 10-30%.

In yet another preferred method, the described compound is produced from borax, dryobalanops aromatica gaertn. f., cortex phellodendri, coptis chinensis franch, baphicacanthus rhizome and root, calculus bovis and moschus moschiferus L. Taking the total weight of the raw materials as 100%, the usage of the different raw materials are as follows: borax 10-70%, dryobalanops aromatica gaertn. f. 5-40%, cortex phellodendri 5-40%, coptis chinensis franch 1-40%, baphicacanthus rhizome and root 5-40%, calculus bovis 3-20%, and moschus moschiferus L. 3-20%; the usage in another preferred method are: borax 30-70%, dryobalanops aromatica gaertn. f. 5-20%, cortex phellodendri 5-20%, coptis chinensis franch 1-20%, baphicacanthus rhizome and root 5-10%, calculus bovis and moschus moschiferus L. 3-10%; the usage in the most preferred method are: borax 40-70%, dryobalanops aromatica gaertn. f. 10-20%, cortex phellodendri 5-10%, coptis chinensis franch 1-10%, baphicacanthus rhizome and root 5-10%, calculus bovis 3-5%, and moschus moschiferus L. 3-5%.

### Medicinal Form

This invention provides the medicinal form of the compound and medically acceptable formatives, for improving brain functioning and/or treatment of brain disorders.

Preferably, the medicinal form would be in powder or fluid form; more preferably in fluid form such as nasal drops or oral drops.

### Preparation of the Compounds

The preparation method of the compounds as described above involves the grinding to powder form and mixing together of the raw materials, extraction by water in the weight ratio of the raw materials to water being 1:20-200; preferably 1:50-150, extraction is done preferably under 90-100 degrees Celcius for 15-120 minutes. After extraction, it is freely filtered and injected into 10 ml bottles for application.

Preferably, the preparation method described includes appropriate grinding to powder form and mixing of the following raw materials: borax, dryobalanops aromatica gaertn. f., cortex phellodendri, coptis chinensis franch, baphicacanthus rhizome and root, and a free selection from calculus bovis, moschus moschiferus L. The powders are then weighed in precision and mixed thoroughly, in the ratio as described in the compounds section above. The thoroughly mixed powder are transferred to the extracting machine, in the ratio of powder to water being 1:20-200 (in weight), more preferably to add pure water in the ratio of 1:50-150, and re-extracted under 90-100 degree Celcius for 15-120 minutes, more preferably for 15-90 minutes, and the most preferred for 15-60 minutes, and then placed in room temperature for cooling, filtered using 200 targeted filters, and injected into 10 ml bottles for use.

Preferably, the resulting fluid medicinal product (drops) will go through tests on its [symbol] , micro-organisms, heavy metal and toxicity, and pesticide residue. The method are detailed below:

### Test on Particle Size

10 ml of the fluid medicinal product (drops) are extracted. After shaking it well, place it on a piece of glass, and examined using 50-100 power microscope. The requirement is that there is no collection of undissolved substances, and no particles greater than 50 μm.

### Test on Micro-Organism

In accordance with the method as described in Appendix XIIIC of the Compendium of Medicine of the People's Republic of China (2005 edition), the sample is tested for the number of bacteria, molds, yeast, and E. Coli. The number of bacteria per ml must not exceed 100, and the number of fungi and yeast per nil must not exceed 100, and no amount of E. Coli. is allowed.

### Test on Heavy Metal and Toxic Elements

The TCM HM1-001 method is used. The sample is disintegrated by acid and tested with Inductively Coupled Plasma-Atomic Emission Spectrometry (ICP-EAS) to examine the content of arsenic, lead, mercury, and cadmium. In the fluid medicinal product, the level of arsenic must not exceed 1500 μg/30 ml, the level of lead must not exceed 179 μg/30 ml, the level of mercury must not exceed 36 μg/30 ml, and the level of lead must not exceed 3500 μg/30 ml.

### Test on Pesticide Residue

The TCM PEST 1-002 method is used. The sample is dissolved in a solvent, and tested using chromatography and mass spectrometry. These include Alrin and Dieldrin, chlordane (cis-, trans-, oxidised chlordane), DDT and derivatives (4,4′-DDT, 2,4-DDT, 4,4′-DDE, 4,4′-DDD), Endrin, heptachlor (heptachlor and heptachlor epoxide), hexachlorobenzene, hexachloro-cyclohexane (except γ-BHC), lindane, pentachloro nitrobenzene (pentachloro nitrobenzene, pentachloroaniline and methyl pentachlorophenyl sulphide). The upper limit for Alrin and Dieldrin is 0.05 mg/kg (millionth percent), chlordane 0.05 mg/kg (millionth percent), DDT and derivatives 1.0 mg/kg (millionth percent), Endrin 0.05 mg/kg (millionth percent), heptachlor 0.05 mg/kg (millionth percent), hexachlorobenzene, 0.1 mg/kg (millionth percent), hexachloro-cyclohexane 0.3 mg/kg (millionth percent), lindane 0.6 mg/kg (millionth percent), and pentachloro nitrobenzene 1.0 mg/kg, (millionth percent).

### Uses

This invention is a compound used as a medicine to improve brain functioning and/or treating brain disorders. These brains disorders include ADHD, Alzheimer's disease, ASD, epilepsy, mental retardation and stroke, etc.

The following are specific examples to illustrate the invention in greater detail.

### Application 1: Preparation of the Compound

To grind into powder borax, chrysanthemum, dryobalanops aromatica gaertn. f., cortex phellodendri, coptis chinensis franch, and baphicacanthus rhizome and root, and mix thoroughly the exact amounts of each as follows: borax powder 40 g, chrysanthemum powder 36 g. dryobalanops aromatica gaertn. f. powder 6 g, cortex phellodendri powder 6 g, coptis chinensis franch 6 g, and baphicacanthus rhizome and root powder 6 g. Transfer the mixture into the extracting machine, and add pure water in the ratio of mixture to water 1:200 (weight), and re-extract under 90-100 degrees Celcius for 1 hour, and cooled at room temperature, filtered using 200 target filters, and inject into 10 ml bottles for use. The fluid drops are then tested for particle size, micro-organisms, heavy metal and toxic elements and pesticide residue. Results indicated that for particle size, there is no undissolved substances and no particles above 50 μm. The results for tests for micro-organism, heavy metal and toxic elements, and pesticide residue are shown below:

| Test | Results | Upper Limit | |
|---|---|---|---|
| Micro-organism | | | |
| Total no. of bacteria | <10/ml | 100/ml | |
| No. of mold & yeast | <10/ml | 100/ml | |
| E. coli. | not detected | 0/ml | |
| Heavy metal and toxic elements | | | |
| Arsenic | <10 μg/30 ml | 1500 μg/30 ml | |
| Lead | <9 μg/30 ml | 179 μg/30 ml | |
| Mercury | <5 μg/30 ml | 36 μg/30 ml | |
| Cadmium | <0.6 μg/30 ml | 3500 μg/30 ml | |
| Pesticide residue | | | |
| Alrin & Dieldrin | <0.02 mg/kg | 0.05 | mg/kg |
| Chlordane | <0.02 mg/kg | 0.05 | mg/kg |
| DDT and derivatives | <0.05 mg/kg | 1.0 | mg/kg |
| Endrin | <0.02 mg/kg | 0.05 | mg/kg |
| Heptachlor | <0.02 mg/kg | 0.05 | mg/kg |
| Hexachlorobenzene, | <0.01 mg/kg | 0.1 | mg/kg |
| Hexachloro-cyclohexane | <0.03 mg/kg | 0.3 | mg/kg |
| Lindane | <0.01 mg/kg | 0.6 | mg/kg |
| Pentachloro nitrobenzene | <0.02 mg/kg | 1.0 | mg/kg |

### Application 2: Preparation of the Compound

To grind into powder borax, dryobalanops aromatica gaertn. f., cortex phellodendri, coptis chinensis franch, baphicacanthus rhizome and root, calculus bovis and moschus moschiferus L., and mix thoroughly the exact amounts of each as follows: borax powder 60 g, dryobalanops aromatica gaertn. f. powder 18 g, cortex phellodendri powder 5 g, coptis chinensis franch 2 g, baphicacanthus rhizome and root powder 5 g, calculus bovis powder 5 g, and moschus moschiferus L. powder 5 g. Transfer the mixture into the extracting machine, and add pure water in the ratio of mixture to water 1:50 (weight), and re-extract under 90-100 degrees Celcius for 1.5 hour, and cooled at room temperature, filtered using 200 target filters, and inject into 10 ml bottles for use.

Application 3: The Compound in this Invention Increases Norepinephrine in the Brain

Six experimental mice receive the compound described in Application 2 above, and another six mice receive saline solution. 40 μl of the medicinal compound was applied through the nasal cavity. After one hour, the brains of the mice are extracted and smeared, and extracted through dissolving in 0.1 ml/L HClO4 and 0.2 mmol/L EDTA—2BA solvent. The level of norepinephrine in the mice's brain smears were measured using the HPLC fluorescent test of the 3.2 chromatography workstation (Waters, USA). The results are as follows:

Chromatographic column: Waters Nova-Pack C18 chromatographic column (150 mm×3.9 mm, 4 μm)

Mobile phase: 14:86 for methanol to buffer solution, in which the buffer solution contains 40 mmol/L of CH3COONa, 30 of citric acid, 0.2 mmol/L of EDA-2Na and 0.4 mmol/L of sodium octanesulphonate, and solvent at pH 3.8:

Fluorescent test wave length: λ_{EX}=280 nm, λ_{EM}=330 nm;

The methanol used was for chromatography, therefore the water used is double-distilled water.

The test results are shown in Table 1, results indicated that the level of norepinephrine in the brain of the mice receiving the invention compound (mean: about 88 pg/mg of tissue) was 5 times higher than those receiving saline solution (mean: about 17 pg/mg of tissue) (p<0.01), indicating that this compound is effective in increasing the level of norepinephrine in the brain.

TABLE 1

Brain norepinephrine level (pg/mg of tissue)

| Mice | Saline solution | Invention compound |
|---|---|---|
| 1 | 4.53 | 156.06 |
| 2 | 15.49 | 47.15 |
| 3 | 22.43 | 94.99 |
| 4 | 13.91 | 52.34 |
| 5 | 17.08 | 53.42 |
| 6 | 28.67 | 124.46 |
| Mean | 17.01 | 88.07 |

Application 4: The Compound in this Invention has a Dose-Response Effect on Increasing the Level of Norepinephrine in the Brain

14 experimental mice are administered the compound described in Application 2, and another 7 were administered saline solution. 40 μl of the medicinal compound was applied through the nasal cavity, twice a day, with the total dosage of 80 μl. After 3 days of application, 7 of the 14 mice receiving the compound were killed on the 4^{th} day, and their brains extracted, smeared and frozen at −60 degrees Celcius for later use. After continuous application of the medicinal compound, the remaining 7 mice receiving the compound and the 7 mice receiving saline solution were killed on the 8^{th} day, with their brains extracted and smeared. The HPLC fluorescent method was used to test the level of norepinephrine in the mice's brains, and the results are shown in Table 2. Results indicated that the mice receiving 7 days of the medicinal compound had higher levels (p<0.001) of norepinephrine in the brain (mean: 46.10 pg/mg of tissue) than those receiving saline solution (mean: 22.03 pg/mg of tissue); the mices that received 3 days of the medicinal compound also had higher level of norepinephrine in the brain (mean: about 33 pg/mg of tissue) than those receiving saline solution (p<0.001). These results suggested that the medicinal compound has a dose-response effect on increasing the levels of norepinephrine in the brain.

TABLE 2

| | Brain norepinephrine level (pg/mg of tissue) | | |
|---|---|---|---|
| | | Medicinal compound | Medicinal compound |
| Mice | Saline solution | (3 days) | (7 days) |
| 1 | 27.89 | 32.47 | 41.52 |
| 2 | 20.78 | 36.24 | 45.47 |
| 3 | 12.67 | 31.83 | 49.69 |
| 4 | 29.53 | 42.15 | 58.95 |
| 5 | 24.47 | 30.25 | 34.94 |
| 6 | 24.99 | 24.53 | 51.75 |
| 7 | 13.84 | 33.87 | 40.40 |
| Mean | 22.03 | 33.05 | 46.10 |

Application 5: The Compound in this Invention has a Dose-Response Effect on Increasing the Level of Norepinephrine in the Brain

Six healthy adults aged between 26 to 42 years were administered 10 ml of the compound described in Application 1 above. After half an hour, their brain activities were measured, using a method developed by Prof Leuchter of the University of California (see Electroencephalography & Clinical Neurophysiology, 1998, 107, 408-414). The absolute and relative power of the brain were used to estimate brain activity. This measure was shown to be correlated with brain perfusion. Results are shown in Table 3. Results indicated that brain activity increased 3-fold after administration of the medicinal compound, corresponding to a large effect size of 0.91 (effect sizes from 0.8 to 1.0 are considered large), indicating that this compound is effective in increasing brain activity.

TABLE 3

Brain activity (z-value)

| Participant | Before | After |
|---|---|---|
| 1 | −0.47 | 0.25 |
| 2 | 0.10 | 0.03 |
| 3 | −0.81 | 1.50 |
| 4 | −0.15 | 0.43 |
| 5 | 1.03 | −0.46 |
| 6 | −1.32 | −0.46 |
| Mean | −0.27 | 0.50 |

### Application 6: In Vitro Test

An individual, aged 20, having become intellectually impaired due to epilepsy. Before administration of the medicinal compound, he cannot comprehend conversations and lost his language ability. 30 ml of the medicinal compound was administered through the nose daily. After 3 weeks, the patient was able to comprehend and produce simple sentences, and his attention was improved. He was able to converse with others in 1 to 3 sentences.

1. A compound for improving brain functioning and/or treatment of brain disorders, with the following raw materials:
(1) One or more medicinal herbs from borax, radix isatidis, honeysuckle, fritillaria cirrhosa D. Don and thunbergii;
(2) One or more medicinal herbs from dryobalanops aromatica gaertn. f., calculus bovis, moschus moschiferus L., liquidambar orientalis mill and gum benzoin;
(3) One or more medicinal herbs from cortex phellodendri, coptis chinensis franch, scutellaria baricalensis, radix gentianae, sophora flavescens;
(4) One or more medicinal herbs from baphicacanthus rhizome and root, calculus bovis, indigowoad leaf, taraxacum officnala, rhizome of the manyleaf Paris, wild chrysanthemum; and the free selection of
(5) Chrysanthemum, Folium Mori, mint, arctium lappa.

2. In the compound from 1. above, with the total weight of raw materials (1) to (5) as 100%, the respective percentage weight of the raw materials (1), (2), (3), (4) and (5) are 10-80%, 3-40%, 3-40%, 3-40%, and 0-50%.

3. The compound from 1. or 2. above are manufactured from borax, dryobalanops aromatica gaertn. f., cortex phellodendri, coptis chinensis franch and baphicacanthus rhizome and root.

4. The compound from 3. above, with the total weight of all raw materials as 100%, the usage for borax is 50-80%, dryobalanops aromatica gaertn. f. 7-20%, cortex phellodendri 5-20%, coptis chinensis Franch 3-15%, baphicacanthus rhizome and root 3-15%.

5. The compound from 1. or 2. above are manufactured from borax, dryobalanops aromatica gaertn. f., cortex phellodendri, coptis chinensis franch, baphicacanthus rhizome and root and chrysanthemum.

6. The compound from 5. above, with the total weight of all raw materials as 100%, the usage of borax is 20-60%, dryobalanops aromatica gaertn. f. 5-20%, cortex phellodendri 5-20%, coptis chinensis franch 5-20%, baphicacanthus rhizome and root 5-20%, chrysanthemum 10-50%.

7. The compound from 1. or 2. above are manufactured from borax, dryobalanops aromatica gaertn. E, cortex phellodendri, coptis chinensis franch, baphicacanthus rhizome and root, calculus bovis, and moschus moschiferus L.

8. The compound from 7. above, with the total weight of all raw materials as 100%, the usage of borax is 30-70%, dryobalanops aromatics gaertn. f. 5-20%, cortex phellodendri 5-20%, coptis chinensis franch 1-20%, baphicacanthus rhizome and root 5-10%, calculus bovis 3-10%, moschus moschiferus L. 3-10%.

9. The medicinal product for improving brain functioning and/or treatment of brain disorders, the specifications include any one compound from 1. to 8. above and medically acceptable formatives.

10. The medicinal preparation in 9. above is in the form of fluid.

11. The fluid as stated in the medicinal preparation in 10. above is in the form of nasal drops.

12. The fluid as stated in the medicinal preparation in 10. above is in the form of oral drops.

13. The preparation method of the compounds in 1. to 8. above involves the grinding to powder form and mixing together of the raw materials, extraction by water, and filtered. The weight ratio of the raw materials and water is 1:20-200.

14. The preparation method as stated in 13. above is done under the temperature of 90-100 degrees Celcius.

15. The compounds in 1. to 8. above are produced for use in improving brain functioning and/or treatment of brain disorders.

16. The use for treatment of brain disorders as stated in 15. above includes attention deficit/hyperactivity disorder (ADHD), Alzheimer's disease, autism spectrum disorders (ASD), epilepsy, mental retardation and stroke.
